(19) United States
(12) Patent Application Publication
(10) Pub. No.: US 2015/0114404 A1
(43) Pub. Date: Apr. 30, 2015

(54) STERILE DRAPE FOR AN O-SHAPED CT SCANNER AND METHOD OF DRAPING AN O-SHAPED CT SCANNER

(71) Applicant: Contour Fabricators, Inc., Fenton, MI (US)

(72) Inventors: Michael W. Czop, Fenton, MI (US); Samba Toure, Grand Blanc, MI (US)

(21) Appl. No.: 14/530,549

(22) Filed: Oct. 31, 2014

Related U.S. Application Data
(60) Provisional application No. 61/898,319, filed on Oct. 31, 2013.

(51) Int. Cl. A61B 19/08; A61B 19/12

(52) U.S. Cl. CPC A61B 19/081; A61B 19/12
USPC 128/856

(57) ABSTRACT

A sterile drape for an o-shaped CT scanner and method of draping an O-shaped CT scanner therewith is provided. The sterile drape has a sterile, flexible tubular wall extending between opposite first and second ends. The first end is open and the second end is substantially closed. The wall has a first seam adjacent the second end. The first seam extends along a circumference of the wall to facilitate forming an opening in the wall adjacent the second end to allow the CT scanner to be fully closed through the opening.

### CROSS-REFERENCE TO RELATED APPLICATION

This application claims the benefit of U.S. Provisional Application Ser. No. 61/898,319, filed Oct. 31, 2013, which is incorporated herein by reference in its entirety.

### BACKGROUND OF THE INVENTION

1. Technical Field

This invention relates generally to sterile surgical drapes, and more particularly to sterile surgical drapes for o-shaped computed tomography (CT) scanners.

2. Related Art

The risk of infection to a patient within a surgical theatre by the transfer of bacteria, such as bacteria from a person or surgical equipment, is a well-known, recurring problem. To minimize the risk of infection during a surgical procedure, it is paramount to prevent the transfer of bacteria, which can be transferred via airborne lint or dust particles, fluids, or otherwise, within the surgical theatre. Infections are estimated to affect about 2 million patients annually and result, directly or indirectly, in an estimated 100,000 deaths. Aside from the loss of life, the infections create an economic burden on hospitals. For example, some studies estimate that each bloodstream infection in a patient results in an average cost of about $27,000 to the hospital. As such, to date, about 16 states have passed laws detailing how hospitals treat patients in an effort to decrease the associated risks of infection to the patients.

During a surgical procedure, it is known to use fluoroscopy imaging devices to allow a surgeon to see real-time internal images of a patient to facilitate the ability of the surgeon to perform the procedure. One common type of fluoroscopy imaging equipment used in surgical procedures is an o-shaped CT scanner, such as can be purchased under the registered trademark (O-arm®) from Medtronic, by way of example. The o-shaped CT scanner has a gantry that is initially open in the general shape of a C prior to use, which allows the scanner to be positioned about the prostrate patient, and then, a moveable arcuate portion of the scanner can be telescopically extended to close off the open section of the scanner, thereby providing the scanner with a continuous, annular or o-shaped gantry.

A drape is known for use with o-shaped CT scanners, however, the known drape has drawbacks that can promote the unwanted transfer of bacteria, which, as discussed above, can result in infection. Further, the known drape typically requires the continual presence of at least 2 people for deployment, and thus, can present potential problems if 2 persons are continuously available. For example, the known drape has a tubular wall extending between opposite open ends, wherein both ends remain open “as packaged” and “as used” throughout the entire deployment. With both ends remaining open, bacteria are free to be transferred into and out from the tubular wall of the drape. In addition, with both ends being open during deployment, one person must remain at each end throughout the entire deployment to ensure the drape extends as intended as the moveable section of the scanner is telescopically extended.

A drape constructed in accordance with the invention overcomes at least the aforementioned limitations, and provides further benefits as will become apparent to one possessing ordinary skill in the art upon viewing the disclosure herein.

### SUMMARY OF THE INVENTION

In accordance with one aspect of the invention, a sterile drape for an o-shaped CT scanner is provided. The sterile drape has a sterile, flexible tubular wall extending between opposite first and second ends. The first end is open and the second end is substantially closed. The wall has a first seam adjacent the second end. The first seam extends along a circumference of the wall to facilitate forming an opening in the wall adjacent the second end to allow the CT scanner to be fully closed through the opening.

With the second end being substantially closed initially, the ability to maintain sterility about the CT-scanner and within the sterile surgical environment is enhanced, and with the first seam facilitating the formation of an opening, a moveable section of a gantry of the CT-scanner can be moved through the opening to a fully closed position.

In accordance with one aspect of the invention, wall has a second seam between the first seam and the first end. The second seam extends along the circumference of the wall to facilitate tearing a tubular section of the wall, extending from the second seam to the second end, from another tubular section of the wall, extending from the second seam to the first end. To further enhance maintaining sterility within the surgical environment, the second seam is preferably concealed from direct exposure to the external environment by an overlying portion of the flexible tubular wall. The overlying portion can be formed from a portion of a circumferentially extending, generally z-shaped fold section of the flexible tubular wall, wherein selectively releasable fasteners can be provided to releasably fix the overlying portion in the z-shape folded configuration.

In accordance with another aspect of the invention, a first adhesive member can be provided adjacent the first seam to facilitate fixing the first end to a portion of the scanner, which in turn, aside from maintaining the first end in a fixed, non-obstructive location during the imaging procedure, can facilitate retracting the moveable section of the gantry after the imaging procedure without the need for a person to hold the first end within the non-sterile zone.

In accordance with another aspect of the invention, a second adhesive member can be provided adjacent the second seam to facilitate fixing a sterile portion of the drape to a portion of the scanner upon retracting a moveable portion of the scanner after the imaging procedure.

In accordance with another aspect of the invention, a method of draping an O-shaped CT scanner is provided. The method includes the following: disposing an open first end of a flexible tubular wall over a fixed section of a gantry of the O-shaped CT scanner, located within a sterile zone above a surgical table patient support surface; releasably fixing the open first end of the flexible tubular wall to a portion of the O-shaped CT scanner; extending a moveable section of the gantry outwardly from the fixed section and bringing an end of the moveable section into close proximity with a closed second end of the flexible tubular wall within a non-sterile zone beneath the surgical table patient support surface; forming an opening in the flexible tubular wall adjacent the closed second; and extending the moveable section of the gantry through the opening to form a closed loop gantry.

In accordance with another aspect of the invention, the method can further include tearing a perforated seam adjacent the closed second end to form the opening, wherein the perforated seam facilitates the quick and easy formation of a cleanly formed opening.

In accordance with another aspect of the invention, the method can further include retracting the moveable section of the gantry from the non-sterile zone to the sterile zone and tearing a first tubular section of the flexible tubular wall extending within the non-sterile zone from a second tubular section of the flexible tubular wall extending within the sterile zone, and further, maintaining the non-sterile first tubular section within the non-sterile zone. As such, the non-sterile first tubular section is kept from being brought into the sterile zone, thereby enhancing the ability to maintain sterility within the sterile zone.

In accordance with another aspect of the invention, the method can further include unfolding an accordion-folded section of the flexible tubular wall to expose an underlying tear seam and tearing the first tubular section from the second tubular section along the tear seam. With the tear seam underlying a portion of the wall until a selected time, the tear seam is protected against unwanted exposure to potential contamination.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1 is a side view of an o-shaped CT scanner shown with a sterile drape constructed in accordance with the invention fully deployed about a gantry of the o-shaped CT scanner;

FIG. 2 is a perspective view of the o-shaped CT scanner shown in an open, non-draped state;

FIGS. 3A-3H show a progression of steps taken to deploy the drape about the gantry of the o-shaped CT scanner;

FIGS. 3I-3P show a progression of steps taken to allow the o-shaped CT scanner to be removed from a patient;

FIG. 4 is a plan view of the drape shown in an unfolded state; and

FIG. 4A is a cross-sectional view taken generally along the line 4A-4A of FIG. 4.

### DETAILED DESCRIPTION OF PRESENTLY PREFERRED EMBODIMENTS

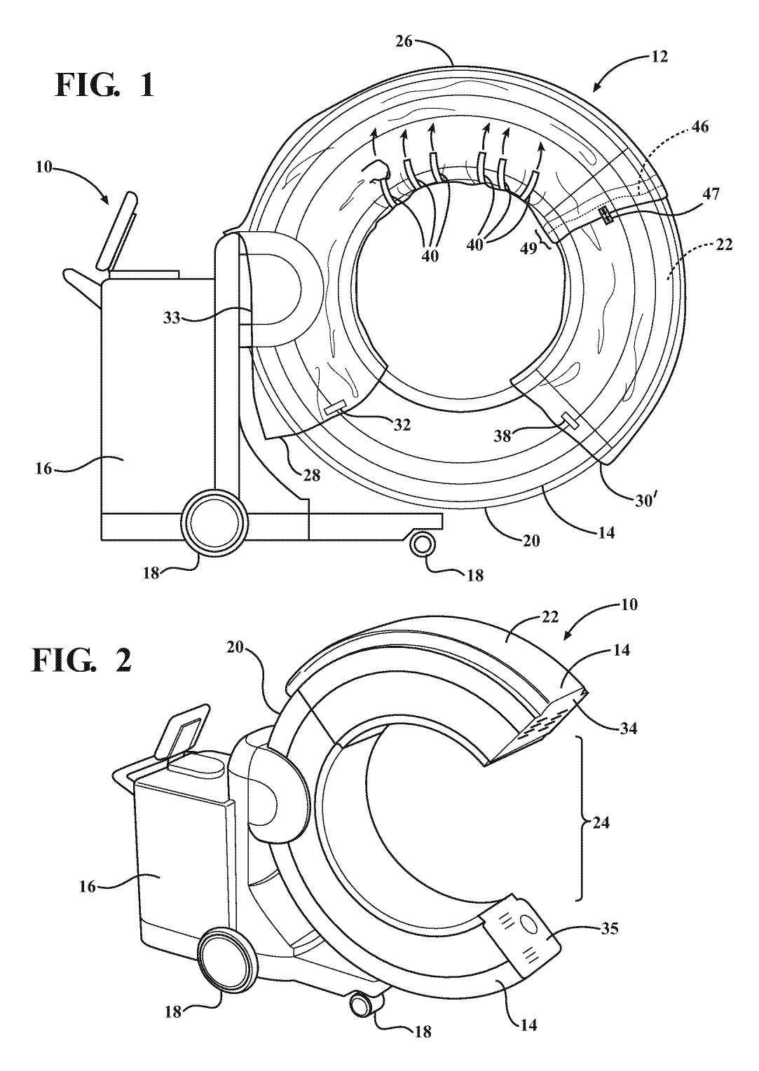

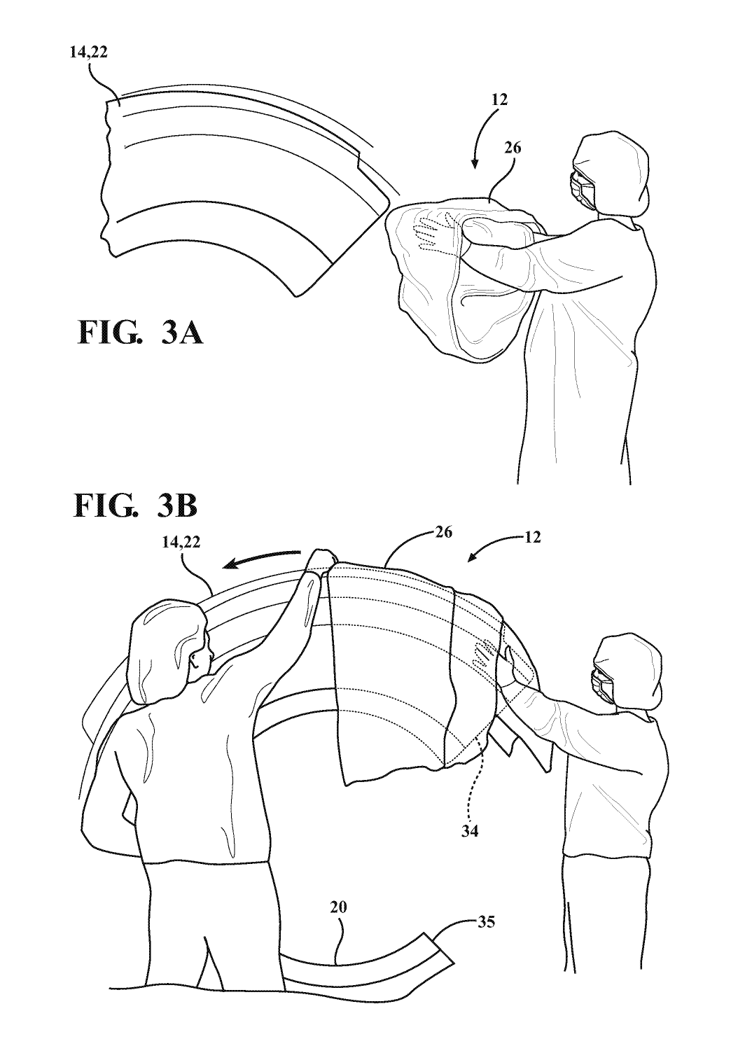

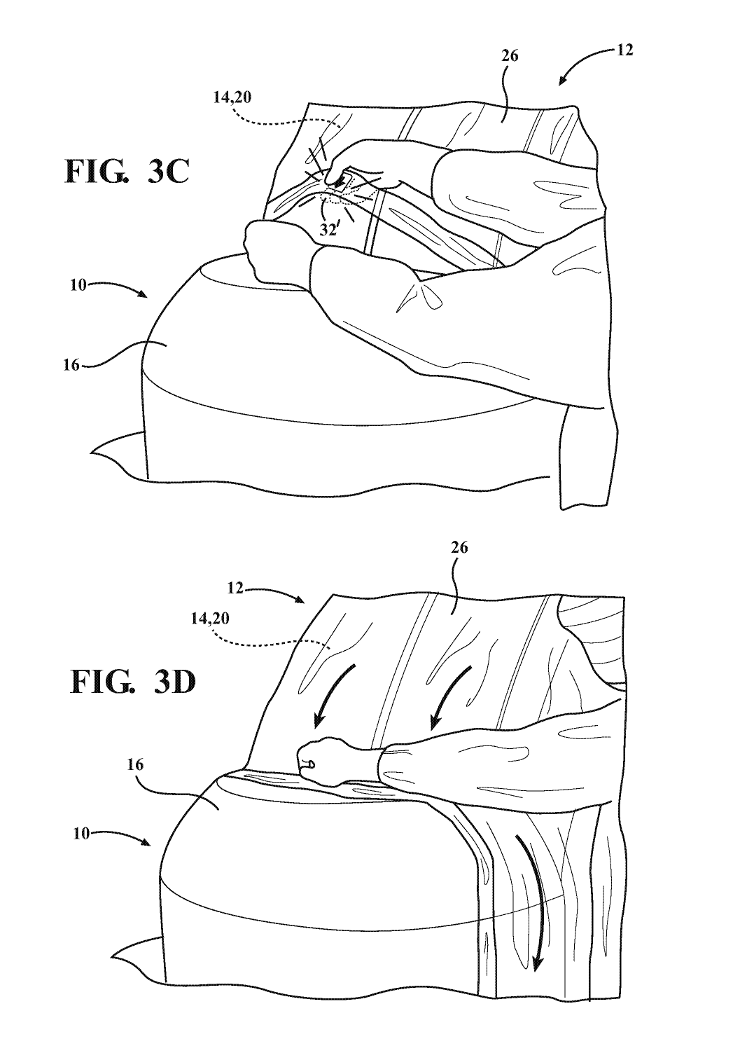

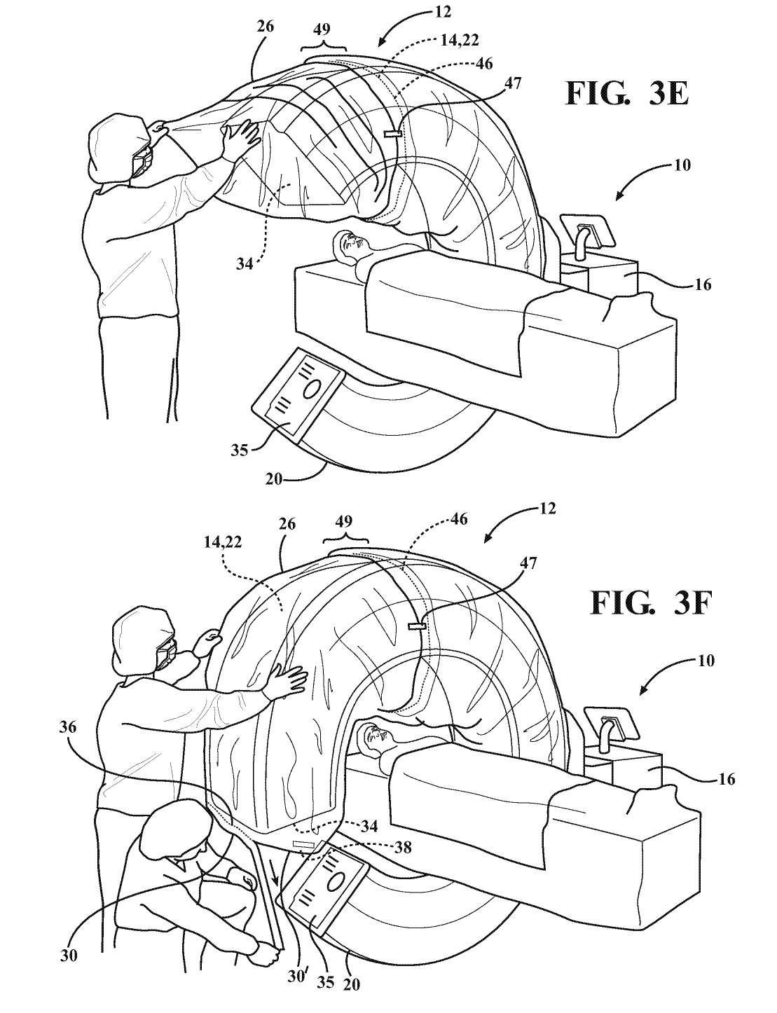

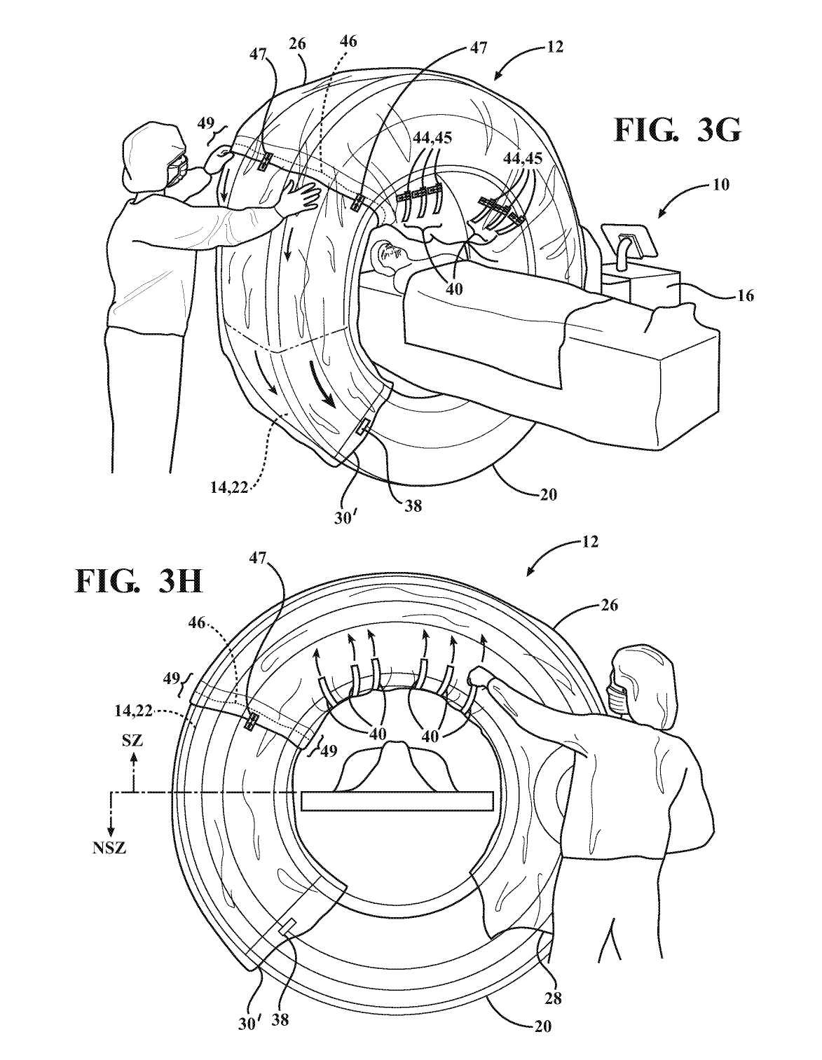

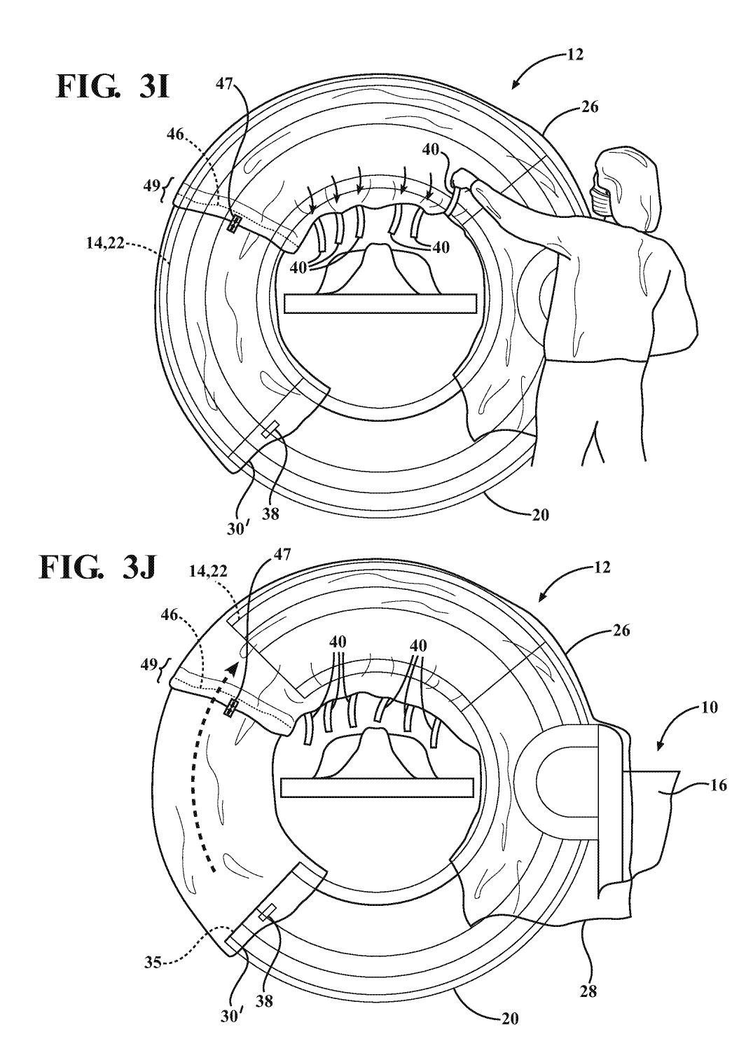

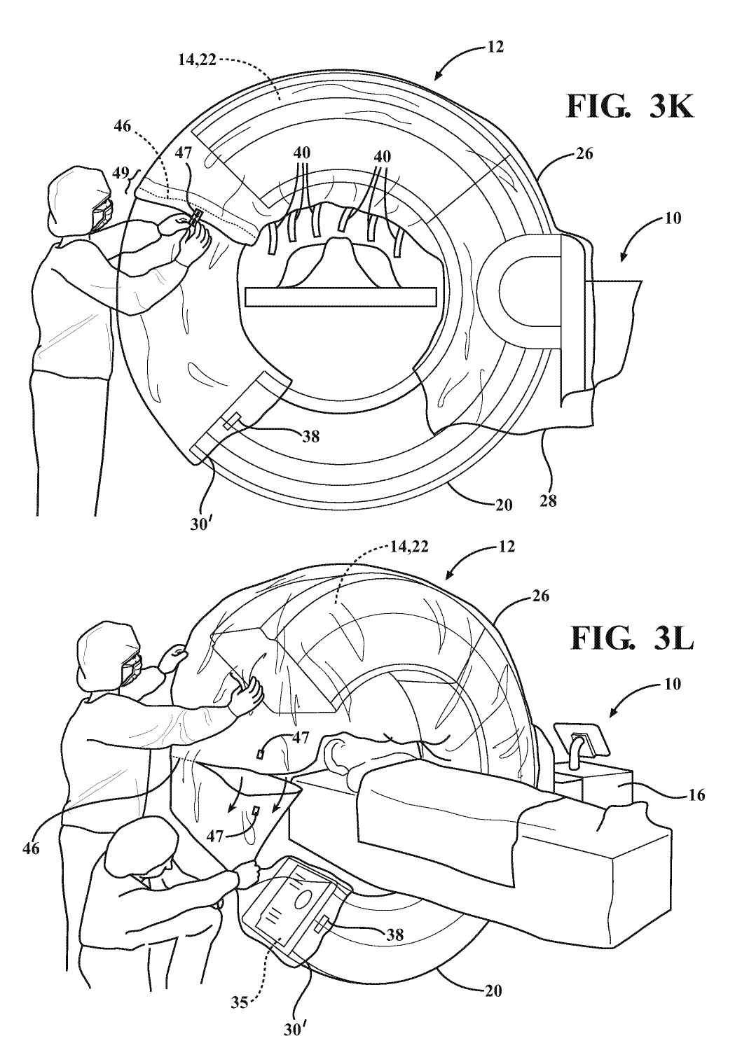

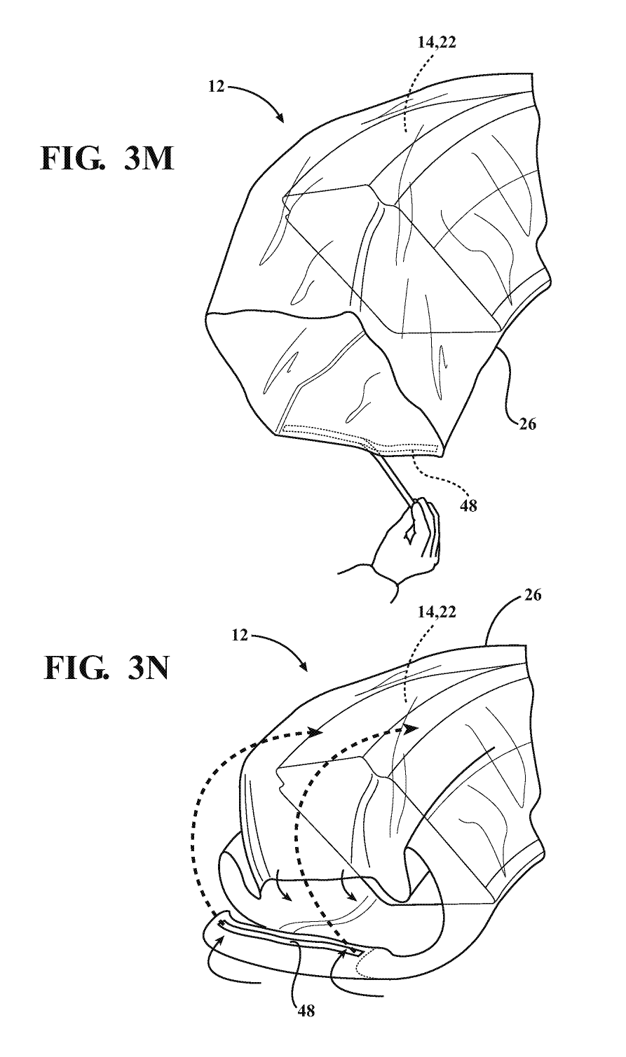

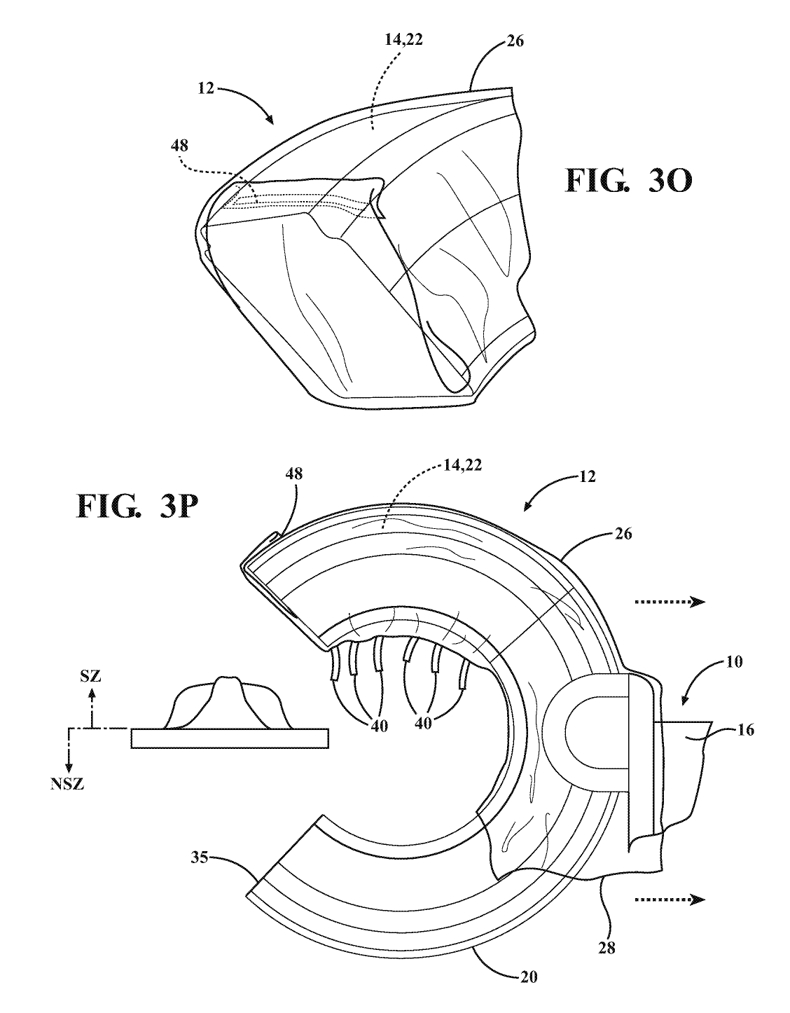

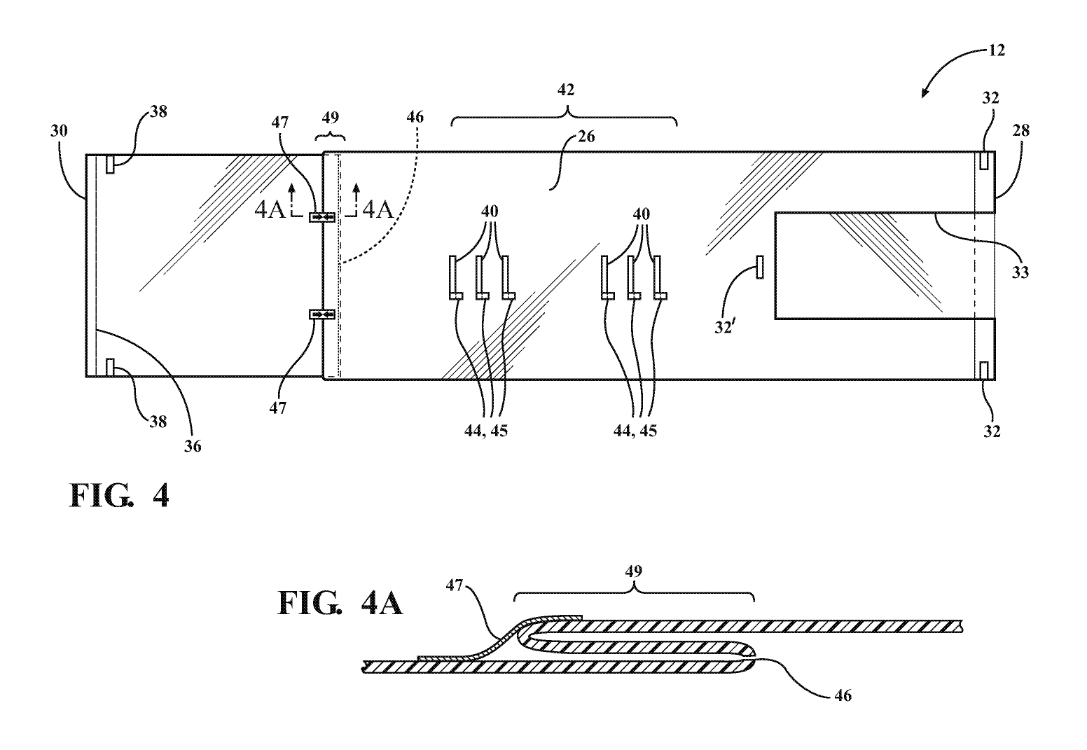

Referring in more detail to the drawings, FIG. 1 illustrates an o-shaped computed tomography (CT) scanner 10 with a sterile drape 12 constructed in accordance with the invention fully disposed thereon to maintain sterility about a patient to be imaged. The drape 12 provides an efficient and reliable way in which to cover an imaging gantry, referred to hereafter simply as gantry 14, of the scanner 10. Further, the drape 12 provides a way in which to best ensure sterility is maintained in a sterile zone SZ (FIG. 3H; area above the support surface of an operating table typically defines a sterile zone in a surgical theatre) of a surgical theatre is maintained, while also ensuring portions of the gantry 14 and the drape 12 lowered into a non-sterile zone NSZ (FIG. 3H; area below the support surface of an operating table typically defines a non-sterile zone in a surgical theatre) of the surgical theatre are prevented from contaminating the sterile zone SZ upon removal of the scanner 10 from an in-use position about the patient. With reference to FIGS. 1 and 2, the scanner 10, as is well known, includes the gantry 14 supported by a base 16, wherein the base 16 is supported for selective movement via wheels 18. The gantry 14 includes a fixed section 20 and a moveable section 22. As shown in FIG. 2, the moveable section 22 is retractable to an open position to form an opening 24, thereby allowing the gantry to be moved into and out of position relative to a patient. As shown in FIG. 1, the moveable section 22 is also moveable to a closed position to close-off the opening 24, thereby forming the generally o-shaped configuration of the gantry 14, which corresponds to the in-use, imaging position of the gantry 14. Prior to using the scanner 10, the drape 12 is partially deployed about the gantry 14 while in its open position (FIGS. 3A-3E), and then, the gantry 14 is moved to its closed position as the drape is fully deployed to shield the patient from potential bacterial contamination (FIGS. 3F-H). The movement of the moveable section 22 during deployment, as discussed further hereafter, can facilitate deploying the drape 12, which can allow a single person, if desired, to more readily deploy the drape 14 about the gantry 14, thereby simplifying the deployment of the drape 12.

The drape 12 has a flexible tubular wall 26 extending between opposite first and second ends 28, 30. As initially constructed and packaged, the first end 28 is open and the second end 30 is closed. As shown, in FIGS. 3A-3B, the open first end 28 is initially disposed about the moveable section 22 while the gantry 14 is in its open state, whereupon the open first end 28 is maneuvered completely over the moveable section 22 and along a portion of the fixed section 20, whereupon the first end 28 is releasably fixed in its intended location via at least one or a plurality of adhesive strips 32 (initially covered with release paper), such as to a portion of the base 16 and/or to a location on the fixed section 20 of the gantry 14. As best shown in FIGS. 1 and 4, to facilitate draping the first end 28 about the base 16, the first end 28 can be provided with a recessed or cut-out region 33, wherein a fastener 32′ can be provided along an edge of the cut-out region 33 to further facilitate releasable fixing the first end 28 of the drape 12 in its intended location.

Upon unfolding and fixing the first end 28 in its intended location, the moveable section 22 of the gantry 14 can be telescopically moved toward the closed state, wherein a free end 34 of the moveable section 22 can abut the closed second end 30 of the drape 12, thereby causing the drape 12 to automatically unfold telescopically toward its fully deployed state. Otherwise, it should be recognized that the drape 12 can be unfolded prior to telescopically extending the drape 12, if desired. Prior to the gantry 14 being fully telescopically extended to its fully closed state, as shown in FIG. 3F, the closed second end 30 is selectively opened to allow the free end 34 of the moveable section 22 to be moved through the established opening into its fully closed state in abutment with a free end 35 of the fixed section 20. To facilitate forming the opening in the closed second end 30, a first perforated seam 36 (FIGS. 3F and 4) is provided adjacent the closed second end 30, wherein the first perforated seam 36 allows the opening to be readily formed, such as by tearing off a narrow end section of the tubular wall 26 immediately adjacent the closed second end 30, thereby forming a new free end 30′ immediately adjacent the location of the removed free end 30. Of course, it should be recognized that an opening can be formed otherwise, without removing the free end 30, such as by forming the opening adjacent the free end 30 through a slit in the wall of the sleeve 12. Regardless of how the opening is formed, upon forming the opening adjacent the second end 30, the moveable section 22 of the gantry 14 can be fully closed (FIG. 3G), and then, the material immediately adjacent the second end 30, 30′ can be releasably fixed to the gantry 14 via at least one or a plurality of adhesive strips 38 (initially covered with release paper), such as to a portion of the fixed section 20. It should be recognized that the adhesive strips 38 are preferably located on an inner surface of the drape wall to facilitate their being fastened without having to invert the drape wall. Accordingly, the drape 12 is releasably fixed to the gantry 14 until it is desired to selectively remove the drape 12 therefrom.

To further facilitate conforming the shape of the drape 12 to the scanner 10, a plurality of gathering strips 40 are preferably provided along a mid-section 42 of the wall 26. The gathering strips 40 can be provided as elongate strips having adhesive pads 44 at one end, wherein the adhesive pads 44 can be initially covered with release paper. The release paper can be selectively peeled off, thereby exposing the underlying adhesive pad 44 for use. To prevent the gathering strips 40 from freely flopping about, the ends with the adhesive pads 44 can be releasably secured to the wall 26, such a via tear strips 45. As such, the tear strips 45 can be selectively torn to allow the gathering strip 40 to be used, as desired. As shown in FIG. 3H, the excess material of the drape 12 can be circumferentially gathered and the adhesive pads 44 can be fixed to a chosen location on the drape wall 26 to releasably maintain the wall 26 of the drape 12 in its gathered, close fit configuration about the gantry 14. It should be recognized that the drape 12, being fastened in a close conforming fit about the mid-section 42 of the gantry 14 via the gathering strips 40, allows full and unfettered use any navigation equipment within the gantry 14, such as a stealth station camera within the gantry 14. Desirably, the gathering strips 40 are positioned a side of the gantry 14 opposite the stealth station camera. By circumferentially gathering the material of the wall 26, the image from the stealth station camera is enhanced, as any wrinkles or folds in the wall material through which the camera must image are removed or substantially removed. Further, the drape 12 can be provided with gathering strips 40 both sides of the drape wall 26, wherein the gathering strips 40 not used can be readily removed to avoid obstructing tracker LED's of the navigation system within the gantry 14.

Upon completing use of the scanner 10, the gantry 14 must be moved to its open configuration in order to remove the scanner 10 from the patient, and thus, the moveable section 22 must be retracted. Prior to retracting the moveable section 22, the gathering strips 40 are detached from their fastened state (Figure I). This allows the moveable section 22 to be freely retracted while the second end 30′ of the drape 12 remains attached to the gantry 14. Accordingly, with the second end 30′ being fastened to the gantry 20, a person is not needed to hold the second end 30′ to prevent the second end 30′ from being raised into the sterile zone SZ while the moveable section 22 of the gantry 14 is being retracted (FIG. 3J). Of course, it should be recognized that, in lieu of the fastener 38, a surgical team member could hold the second end 30′ of the drape 12, if desired, to prevent the portion of the drape 12 hanging within the non-sterile zone NSZ from being lifted into the sterile zone SZ above the surgical table.

Then, as shown in FIGS. 3K and 3L, upon the moveable section 22 of the gantry 14 being retracted to its open position, a tubular section of the wall 26 extending to the second end 30′ is torn from the wall 26 along a second perforated seam 46. The second perforated seam 46 extends circumferentially about the wall 26 between the first perforated seam 36 and the first end 28 of the wall 26, and is shown as being proximate the gathering strips 40 between the second end 30′ and the gathering strips 40. To facilitate maintaining sterility, the second perforated seam 46 is initially covered by an overlying portion of the drape wall 26, wherein the covering portion of the drape wall 26 is folded over the second perforated seam 46 to form z-shape folded region 49, as viewed in lateral cross-section (FIG. 4A). To initially maintain the wall 26 in the z-shaped folded configuration, a plurality of tear fasteners 47 are provided, with opposite ends of the tear fasteners 47 being attached to the wall 26 on opposite sides of the z-shaped folded material, thereby maintaining the wall 26 in its folded configuration. When desired to expose the second perforated seam 46, the tear fasteners 47 are selectively torn (FIG. 3K), thereby allowing the material of the z-shaped fold to be released and extended. As such, the second perforated seam 46 becomes exposed. Then, with the second perforated seam 46 exposed, and with the moveable section 22 in its retracted position, the second perforated seam 46 can be torn to allow the non-sterile tubular section of the drape wall 26 (extending between the second perforated seam 46 and the second end 30′) to be removed from the remaining tubular sterile section of the wall 26 (extending between the second perforated seam 46 and the first end 28).

Upon removal of the non-sterile tubular section of the wall 26 from the remaining sterile tubular section of the wall 26, the non-sterile tubular section is allowed to fall to the floor, thereby remaining within the non-sterile zone NSZ without having been brought into the sterile zone SZ. Then, to further reduce the risk of contaminating the sterile zone SZ and the patient, an adhesive strip 48 immediately adjacent the second perforated seam 46, between the second perforated seam 46 and the first end 28, is unveiled via removal of a release paper (FIG. 3M), and the newly formed open end is sealed or substantially sealed closed via the adhesive strip 48 (FIG. 3N). Preferably, the adhesive strip 48 is on an outer surface of the wall 26, and is folded over to fasten the outer surface of the wall 26 on one side of the new opening to an outer surface of the wall 26 on an opposite side of the new opening. It should be recognized the adhesive strip 48 could be provided as a plurality of adhesive strips, if desired, however, a single adhesive strip extending across the full width or a substantial portion of the width of the wall 26, corresponding to a diameter of the wall 26, allows the full open end of the wall 26 to be sealed off.

Upon sealing off the open end via the adhesive strip 48 and fully covering the end 34 of the gantry 14, the scanner 10 can be removed from its position about the patient without worry of contaminating the patient, and the scanner 10 can be stowed for future use.

Many modifications and variations of the present invention are possible in light of the above teachings. It is, therefore, to be understood that the invention may be practiced otherwise than as specifically described, and that the scope of the invention is defined by any ultimately allowed claims.

What is claimed is:

1. A sterile drape for an o-shaped CT scanner, comprising:
a sterile, flexible tubular wall extending between opposite first and second ends, said first end being open and said second end being substantially closed, said wall having a first seam adjacent said second end, said first seam extending along a circumference of said wall to facilitate forming an opening in said wall adjacent said second end.

2. The sterile drape of claim 1 further including a second seam between said first seam and said first end, said second seam extending along the circumference of said wall to facilitate tearing a tubular section of said wall, extending from said second seam to said second end, from another tubular section of said wall, extending from said second seam to said first end.

3. The sterile drape of claim 2 wherein said second seam is concealed from direct exposure to the external environment by an overlying portion of said flexible tubular wall.

4. The sterile drape of claim 3 wherein said overlying portion is formed as a portion of a circumferentially extending, accordion folded section of said flexible tubular wall.

5. The sterile drape of claim 4 further including selectively releasable fasteners releasably fixing said overlying portion in an accordion folded configuration.

6. The sterile drape of claim 4 wherein said selectively releasable fastener is a tear tab having a perforated tear seam.

7. The sterile drape of claim 2 further including a first adhesive member adjacent said first seam.

8. The sterile drape of claim 7 further including a release paper releasably covering said first adhesive member.

9. The sterile drape of claim 7 wherein said first adhesive member is between said first seam and said second seam.

10. The sterile drape of claim 7 further including a second adhesive member adjacent said second seam.

11. The sterile drape of claim 10 further including a release paper releasably covering said second adhesive member.

12. The sterile drape of claim 10 wherein said second adhesive member is between said second seam and said first end.

13. The sterile drape of claim 2 further including an adhesive member adjacent said second seam.

14. The sterile drape of claim 1 further including an adhesive member adjacent said first seam.

15. The sterile drape of claim 1 wherein said first seam is perforated.

16. The sterile drape of claim 2 wherein said second seam is perforated.

17. A method of draping an 0-shaped CT scanner, comprising:
disposing an open first end of a flexible tubular wall over a fixed section of a gantry of the O-shaped CT scanner, located within a sterile zone above a surgical table patient support surface;
releasably fixing the open first end of the flexible tubular wall to a portion of the O-shaped CT scanner;
extending a moveable section of the gantry outwardly from the fixed section and bringing an end of the moveable section into close proximity with a substantially closed second end of the flexible tubular wall within a non-sterile zone beneath the surgical table patient support surface;
forming an opening in the flexible tubular wall adjacent the substantially closed second end; and
further extending the moveable section of the gantry through the opening to form a closed loop gantry.

18. The method of claim 17 further including tearing a perforated seam adjacent the closed second end to form the opening.

19. The method of claim 17 further including retracting the moveable section of the gantry from the non-sterile zone to the sterile zone and tearing a first tubular section of the flexible tubular wall extending within the non-sterile zone from a second tubular section of the flexible tubular wall extending within the sterile zone.

20. The method of claim 19 further including maintaining the first tubular section within the non-sterile zone.

21. The method of claim 19 further including unfolding an accordion folded section of the flexible tubular wall to expose an underlying tear seam and tearing the first tubular section from the second tubular section along the tear seam.

22. The method of claim 19 further including providing the tear seam as a perforated seam.
